(19) United States
(12) Patent Application Publication
(10) Pub. No.: US 2012/0029484 A1
(43) Pub. Date: Feb. 2, 2012

(54) DEVICE FOR FASTENING METALLIC GUIDES AND BALLOON CATHETERS FOR CARRYING OUT MEDICAL PROCEDURES INVOLVING PERCUTANEOUS VASCULAR SURGERY

(76) Inventors: Marcos de Oliveira Gusmao, Recife - PE (BR); Reginaldo Henrique de Toledo Melcop, Recife - PE (BR)

(21) Appl. No.: 13/146,715

(22) PCT Filed: Jan. 20, 2010
(86) PCT No.: PCT/BR2010/000018

§ 371 Date: Oct. 12, 2011

(30) Foreign Application Priority Data
Jan. 28, 2009 (BR) PI 0900210-3
Jan. 28, 2009 (BR) PI 0900211-1
Jan. 28, 2009 (BR) PI 0900212-0

(51) Int. Cl. A61M 25/18

(52) U.S. Cl. 604/538

(57) ABSTRACT

A device for fastening metallic guides and balloon catheters for carrying out medical procedures involving percutaneous vascular surgery is described. The device may include a flat base with a proximal end and a distal end. Fastening means may be mounted on the tapered proximal end to hold and position a Y connector. At least one pair of aligned fasteners may be mounted on the distal end. Each pair of fasteners may also be aligned with the respective inlets for the main vessel and for the secondary or lateral vessels. Each fastener may have a shape of a truncated pyramid made of a rigid material, may project from the base, and may be provided at the top end with an insert made of a flexible material with a longitudinal groove that defines a receptacle for housing and retaining a metallic guide, a balloon catheter or a balloon catheter with a stent.

### FIELD OF THE INVENTION

The present invention generally refers to the field of surgical interventions, more specifically fixing devices for metal guides and balloon catheters for vascular intervention procedures in medicine.

### BACKGROUND OF THE INVENTION

Percutaneous treatment of obstructions be them simple/angioplasty, bifurcation or trifurcation, is complex, requiring material resources and technical experience, with the use of the following materials being required:
Therapeutic guide catheter, adapted to a valve connector, also known as Y connector, dedicated and specific for angioplasty;
Metal guides, one for the main vessel and eventually others for secondary and side vessels;
Balloon catheters to be passed over metal guides and will be responsible for the dilatation of obstructions or damage;
Balloon catheters with endovascular metal prosthesis, also known as stents, which will be located and released at the level of obstructions or damage after the balloon insufflation or de-insufflation;
Sometimes the use of balloon catheters with larger diameter is required, to improve the expansion of stents, as well as the use of other devices like intravascular ultrasound; and
Devices to measure the extension of damage or obstruction in the vessel, which is an essential parameter to evaluate the size of the stent and/or the balloon to be used. Currently, the most accurate way to measure the extension of damage is by computer, with the support of an expert in the art, but this resource is still very rare. In most cases, said step is subjectively taken, based on a visual analysis by the operator, which may bring in huge mistakes and consequent failure in operation, due to the use of balloons and/or stents with inadequate size.

The success of the skill depends on the precise release of the stent at the level of obstructions or damage. It is important to highlight that said balloon insufflation and de-insufflation may occur isolated or simultaneously.

For balloon catheters and balloon catheters with intravascular prosthesis to slide between each other and over metal guides inside the guiding catheter and the vessels, they need to remain fixed as trails, so that, when one of them moves back or forth, the others do not change their position. Currently, they are manually fixed by the operator, fixing metal guides and one of the balloon catheters with fingers. Therefore, the operator can move only one of these elements at a time, be it the metal guide, the balloon catheter or the balloon catheter with stent, making the procedure take longer and become more difficult.

While the procedure is made, metal guides and balloon catheters are frequently crossed and intertwined, making its movement by the operator become difficult, with the consequent need to take out the guides or balloon catheters for the other materials to slide more easily.

Considering the anatomic complexity of damage, be it simple/angioplasty, bifurcation or trifurcation, linked to the technical difficulty to make the percutaneous treatment, there is also the need to look for enhancements, both in relation to the materials used and aiming to develop devices to make this procedure become easier.

### SUMMARY OF THE INVENTION

It is an object of the present invention to provide a device aiming to facilitate the realization of the percutaneous skill of vascular intervention in the treatment of simple damage/angioplasty, bifurcation or trifurcation, making the procedure become faster, safer, with less exposure to X rays, better possibilities of success and less complications. Therefore, its execution becomes more accessible to most operators, be them skilled or beginners, providing that a larger number of patients may benefit from that skill.

The device to fix metal guides and balloon catheters to perform medical procedures in percutaneous vascular interventions, as per the present invention, comprises a flat base with a proximal end and a distal end, wherein, from said narrowing proximal end, respective fixing means project to support and keep a Y connector in position, and, on said distal end, at least one aligned pair of fixers projects, wherein each pair of fixers is also aligned with respective inlets to the main vessel and to secondary or side vessels.

Each one of the fixers is a trunk-pyramidal structure made of a rigid material, projecting from a base and having, on its upper portion, a graft made of a liable material, which is provided with a lengthwise gutter defining a seat to receive and retain a metal guide, a balloon catheter or a balloon catheter with stent. The device may also comprise at least a millimeter ruler located on the base and the alignment axis between the pairs of fixers.

Various benefits and advantages are obtained with the use of the device of the invention, such as better and easier identification of guides and balloon catheters of the main vessel, the side vessel and the secondary vessel, as per the case, since there will be different inlets by the connector Y. Furthermore, a precise positioning allows for less possibility of crossing and intertwining of metal guides and balloon catheters. There are operational advantageous features, such as quicker and easier positioning and insufflation of balloon catheters and balloon catheters with stents at the level of obstructions; easier removal of balloon catheters; easier passage of another device such as intravascular ultrasound, if required. Another benefit is the use of a millimeter ruler at the level of fixers, which will serve to more quickly, safely and precisely estimate the extension of damage or obstruction than the visual and subjective form. At last, the general result of the procedure is less time for procedure and consequent reduction in the exposure of the patient and the medical staff to X rays; higher probability of success and fewer complications, as well as an increase in the number of patients able to receive said procedure.

### BRIEF DESCRIPTION OF DRAWINGS

FIG. 1 is a perspective view of the device to fix metal guides and balloon catheters of a first way of embodiment of the present invention, to which a connector with valves is coupled, both intended to vascular interventions, mainly for the treatment of bifurcation damage;

FIG. 2 is an upper elevation view of the device of FIG. 1;

FIG. 3 is an upper elevation view of the device of FIG. 1;

FIGS. 4 to 6 are side perspective views showing the steps to fix the valve connector at the proximal end of the device;

FIG. 7 is an upper perspective view of an alternative form of embodiment of the device of the present invention, intended for vascular interventions for the treatment of simple damage;

FIG. 8 is an upper perspective view of more than one alternative form of embodiment of the device of the present invention, intended for vascular interventions for the treatment of trifurcation damage;

FIG. 9 is an upper elevation view of an alternative form of embodiment of the device for the preferential treatment of bifurcation damage, presenting low size with a V discontinuity between the fixers of the main and side vessels; and

FIGS. 10A, 10B and 10C are upper elevation views, similar to the views of FIGS. 1, 7 and 8, of a preferential form of embodiment of the device of the present invention, to which a millimeter ruler is incorporated, aiming to help in estimating the extension of the damage.

### DETAILED DESCRIPTION

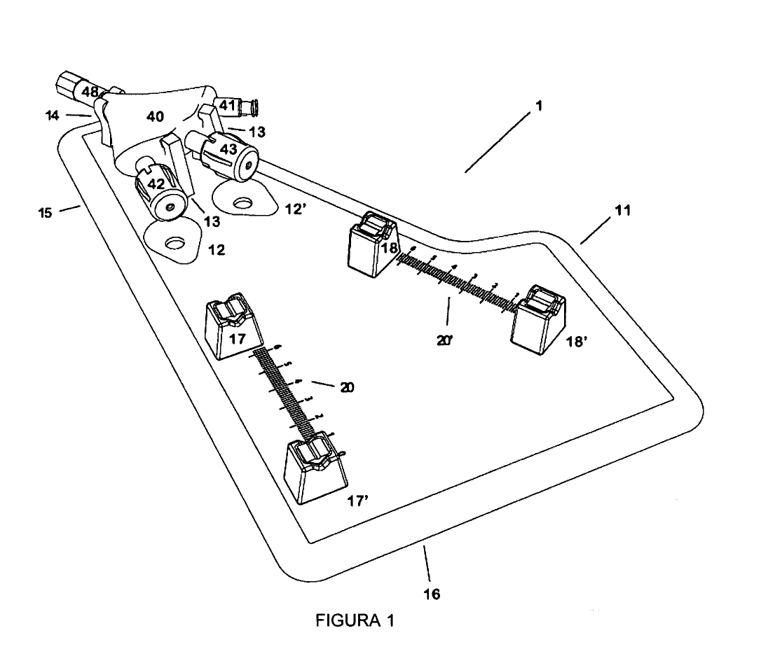

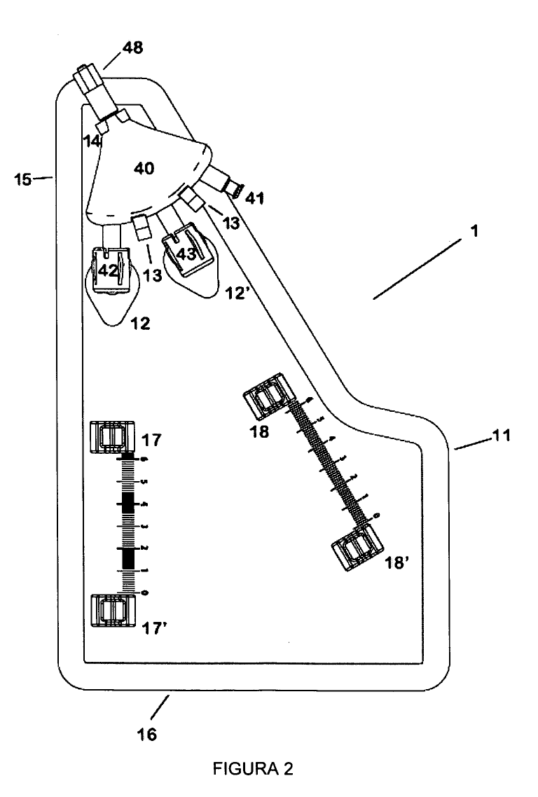

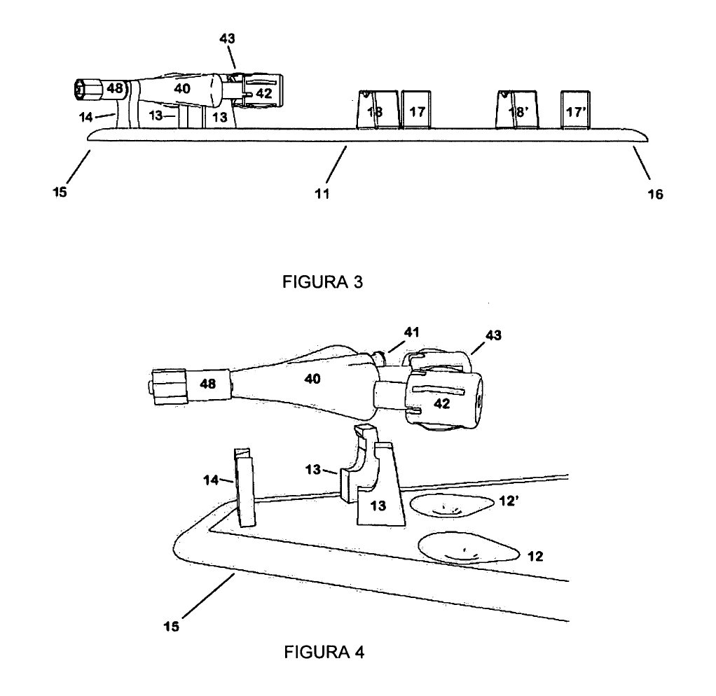

According to the illustrations of FIGS. 1-3, the device 1 to fix metal guides and balloon catheters of the present invention, intended for vascular interventions, mainly to treat damage and bifurcation, is formed by a base or a platform 11 and, on its narrow proximal end 15, has two pins 13 and a jaw 14 to support the Y connector 40 as appropriate for bifurcation, wherein the pins 13 are located so to fix the distal portion of the Y connector 40, while the jaw 14 fixes the proximal portion of said Y connector 40. More specifically, pins 13 are converging prismatic structures projecting from the base 11 and having a recess able to couple the distal portion of the Y connector 40. The jaw 14 is a full prismatic element provided on its upper portion with two opposed holding arms, limiting a seat with similar shape to the external cylindrical shape of the proximal portion of the Y connector 40.

Concerning the valve connector or Y connector 40, this is an element known in the art, intended to direct the guide catheter and, within that and as per the case, the balloon catheter and the balloon catheter with stent over metal guides (not shown), for which reason we do not need to make a detailed description of their operation. In any case, the Y connector 40 has a basically triangular body with rounded edges. From its proximal portion 48, a cylindrical structure is projected to guide and support the elements (balloon catheter, balloon catheter with the stent and metal guides, respectively) to be inserted in the patient by percutaneous route. On its distal or proximal portion, the Y connector 40 has a route 41 to inject contrast agent and determine the intravascular pressure, as well as, in this case, two inlets, of which a first inlet 42 is intended to introduce a metal guide and a balloon catheter (not shown) for the main vessel and the second inlet 43 intended to introduce the metal guide and balloon catheter (not shown) for the secondary or side vessel.

Returning to the device 1 of the invention, below each one of the inlets 42, 43 of the Y connector 40, a respective hole 12, 12′ intended to the flow of blood is located.

Furthermore, on the distal edge 16 of the base 11 of the device 1, preferably wider than the proximal edge 15 of the same base 11, two pairs of fixers 17, 17′ and 18, 18′ are located, wherein the first pair of fixers 17, 17′ is located aligned over the inlet 42 of the Y connector 40 to the main vessel, while the second pair of fixers 18, 18′ is located aligned with the inlet 43 of the Y connector 40 to the secondary or side vessel.

Specifically, any one of the fixers 17, 17′, 18, 18′ is a trunk-pyramidal structure made of a rigid material, projecting from the base 11 of the device 1. On its upper portion, any one of the fixers 17, 17′, 18, 18′ has a graft 5, made of a liable material and provided with a lengthwise gutter 6 composing a seat intended to receive and retain a metal guide, a balloon catheter or a balloon catheter with stent (not shown). Particularly and according to the present invention, to receive means to receive with no lacks and keep it in position not allowing side deflections or displacements, involving or not a given compression of the material composing the gutter 6; while to retain means to keep in position, not allowing movements back and forth of the element as retained over the respective fixer, due to the friction between the internal face of the gutter 6 and the external face of the element to be retained within said fixer. Preferably, the graft 5 has a prismatic shape with crosswise section in half-circle, but, in other embodiments, the crosswise section of the graft 5 may have another appropriate shape.

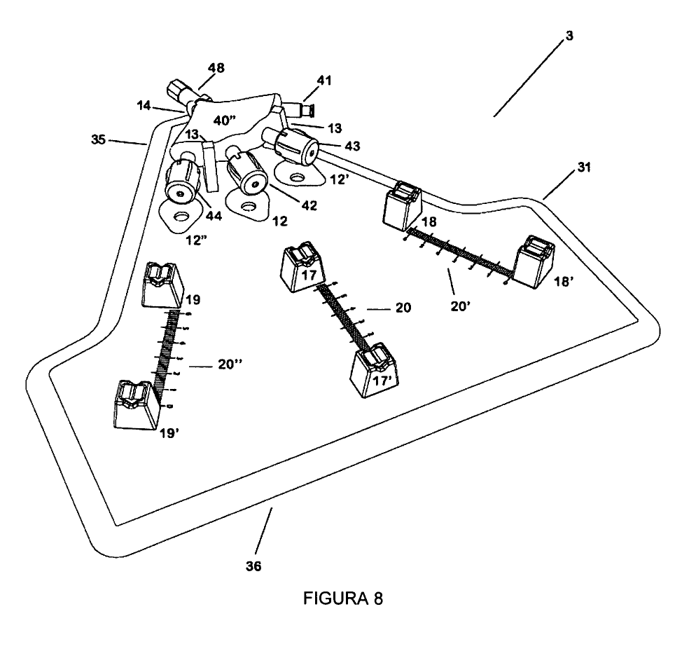

In an alternative form of embodiment of the present invention, as shown by FIG. 7, the device 2 is intended to receive and facilitate the use of a Y connector 40′ intended for use in vascular interventions to treat simple damage. Therefore, said Y connector 40′ has only one inlet 42 intended to introduce a metal guide and a balloon catheter (not shown) for the main vessel, as well as a route 41 for injection of contrast agent and determination of the intravascular pressure.

Specifically, the device 2 of the invention is composed by a base 21 having a narrow proximal end 25 and a distal end 26 opposed to the first one. Similarly to the disclosures above, the proximal end 25 has two pins 13 and a jaw 14 to support the Y connector 40′, wherein the pins 13 are located so to fix the distal portion of the Y connector 40′, while the jaw 14 fixes the proximal portion of said Y connector 40′.

On the distal direction, opposed to the Y connector 40′, four fixers 17, 17′, 17″ and 17″′ are intended, related and aligned to the inlet 42 of the Y connector 40′, being one nearer fixer 17, two intermediate fixers 17′ and 17″ and one fixer 17″′ on the distal portion 26 of the base 21, as opposed to said Y connector 40′. Furthermore, and below the final position taken by the main inlet 42 of the Y connector 40′, an orifice 12 is located, intended to the flow of blood.

In another alternative form of embodiment of the present invention, as shown by FIG. 8, the device 3 is intended to receive and facilitate the use of a Y connector 40″ intended for use in vascular interventions to treat trifurcation damage. Therefore, said Y connector 40″ has a main inlet 42 intended to introduce the metal guide and the balloon catheter (not shown) for the main vessel, two inlets 43 and 44 intended to introduce the metal guide and the balloon catheter in the secondary or side vessels, indistinctively, as well as a route 41 for the injection of contrast agent and determination of intravascular pressure.

The base 31 of the device 3 has a narrow proximal end 35 and a distal end 36 opposed to the first one. Similarly to the disclosures above, the proximal end 35 has two pins 13 and a jaw 14 to support the Y connector 40″, wherein the pins 13 are located so to fix the distal portion of the Y connector 40″, while the jaw 14 fixes the proximal portion of said Y connector 40″.

In the distal direction, opposed to the Y connector 40′, three pairs of fixers 17 and 17′, 18 and 18′, 19 and 19′ are included, being the first pair of fixers 17 and 17′ related and aligned with the main inlet 42 of the Y connector 40″, while the other pairs of fixers 18 and 18′, 19 and 19′ are respectively aligned to each one of the other inlets 43 and 44 for the secondary or side vessels. Furthermore, and below the final position as assumed by each one of the inlets 42, 43 and 44 of the Y connector 40″, respective orifices 12, 12′, 12″ intended for the flow of blood are included.

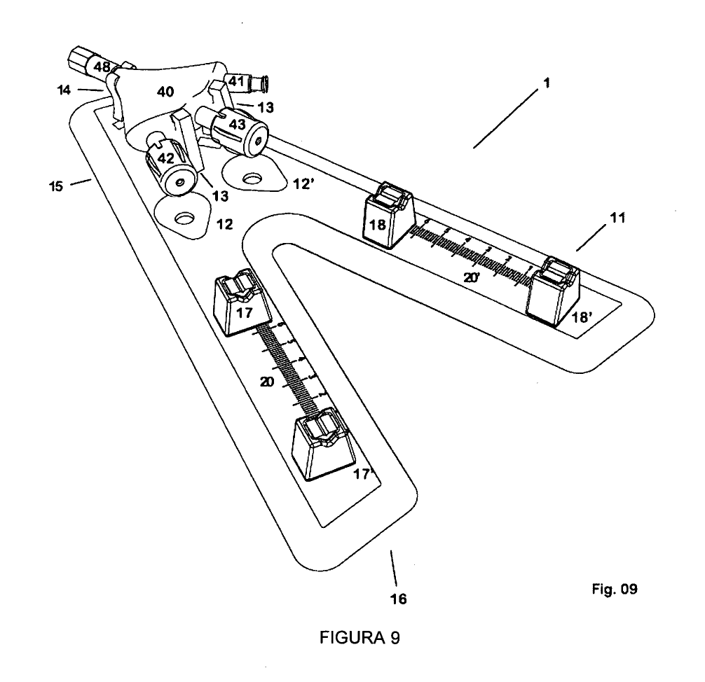

In an alternative form of embodiment, as shown by FIG. 9, the base 11′ of the device 4 to fix metal guides and balloon catheters, intended for vascular interventions, and particularly for the treatment of bifurcation damage, has a V-shaped discontinuity 33 formed between the support regions for the fixers 17, 17′ and 18, 18′. Therefore, each one of said support regions for the fixers 17, 17′ and the fixers 18, 18′ is present in the form of a projection or leg from the central region of the base 11′ of the device 4. Said form of embodiment of the invention allows the quantity of the base material 11′ to be reduced, thus making the device 11′ become lighter and more practical for its use and transport.

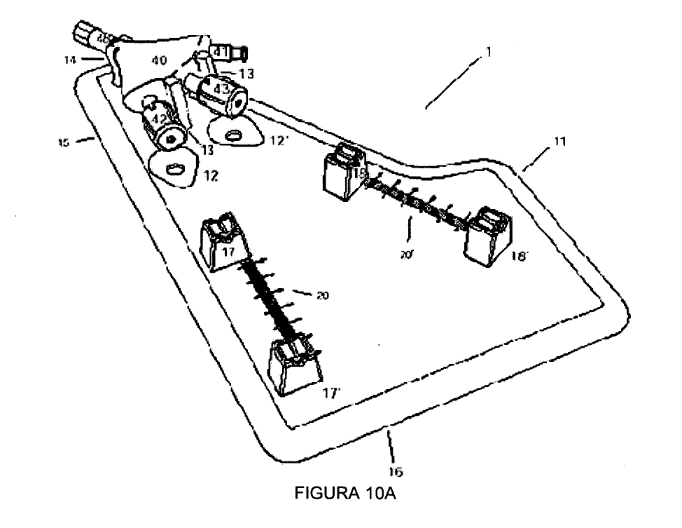

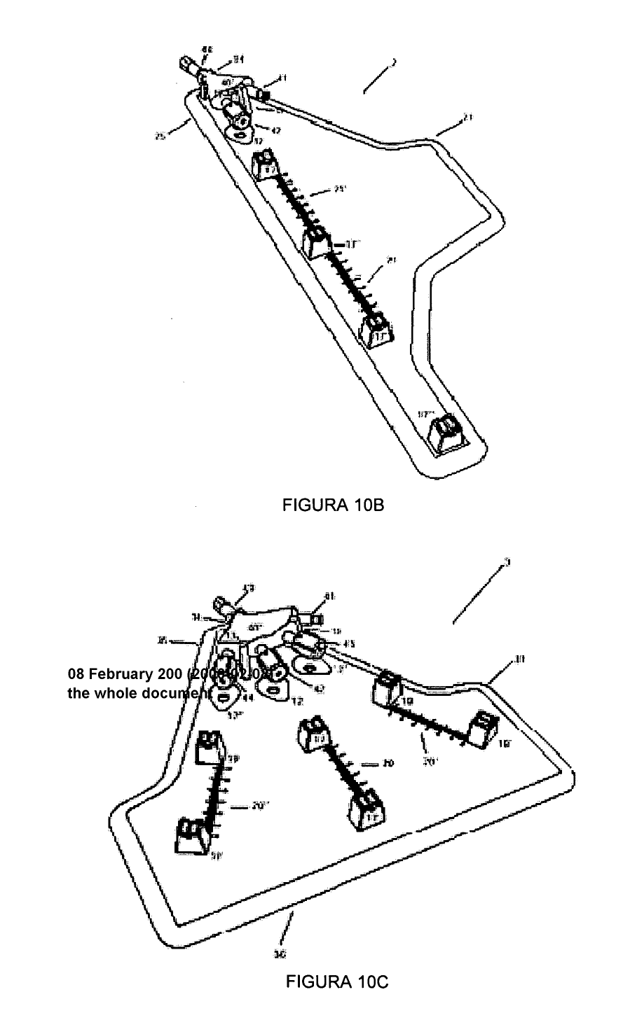

In a preferable way of embodiment of the invention, according to FIGS. 10A, 10B and 10C and so to enable a more precise evaluation of the extension of the damage or obstruction, the line of fixers 17, 17′, 17″, 17″′ includes the disposition and/or printing of a millimeter ruler 20. Said rule 20 may be an element incorporated to the base 11, 11′, 21, 31, but, preferably, said rule 32 is printed over the base 11, 11′, 21, 31. In another possible form of embodiment, the rule 20 may have the form of a sticker with printing over or under a plastic base of a transparent material, as long as the material of the plastic base of the sticker is able to support the device sterilization procedures as required for its use in a surgery room.

The inclusion and disposition of said rule 20 over the base 11, 11′, 21, 31 allows us to evaluate, with relatively higher precision, the effective size of the damage being treated and therefore the size of the balloon and/or stent to be used in the intervention. Alternatively, other millimeter rulers 20, 20′ and 20″ are also located in any one of the bases 11, 11′, 21, 31, respectively the devices 1, 2, 3 or 4, under the axis as defined by the respective pairs of fixers 17 and 17′, 18 and 18′, as well as 19 and 19′.

The object of the present invention will now be disclosed as a function of its practical application, particularly in the percutaneous treatment of bifurcation damage, which is made with the support of the device 1, as shown by FIGS. 1-3. However, we should highlight that the use of embodiments as shown by FIGS. 7 and 8 is similar, with the only variation in the number of metal guides, balloon catheters and/or balloon catheters with stent (not shown) as a function of the type of damage to be treated (angioplasty or trifurcation, respectively).

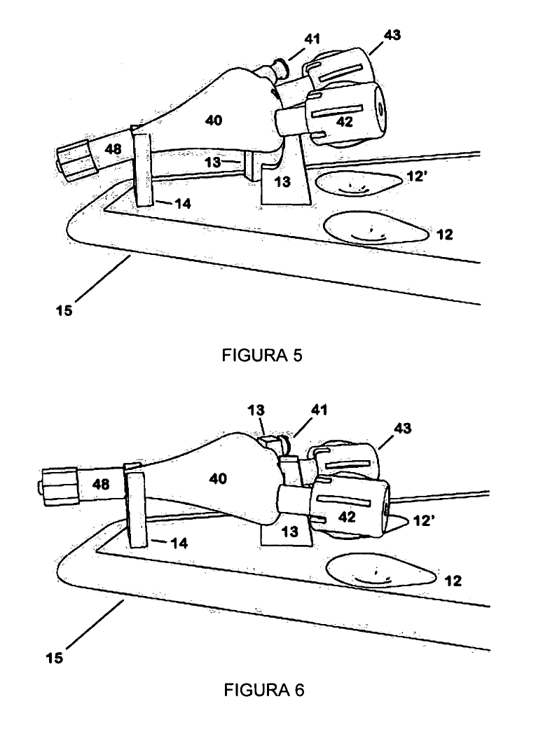

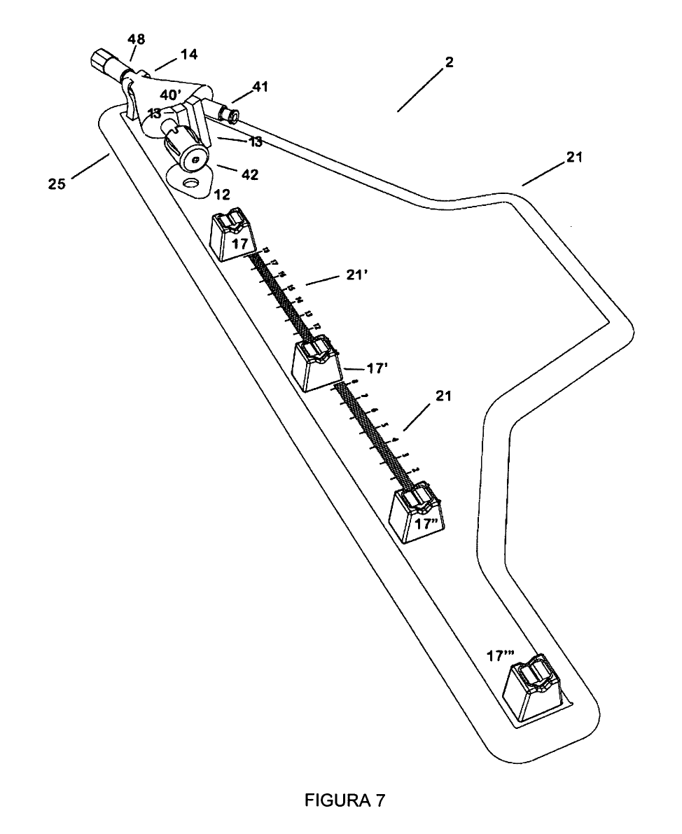

In the percutaneous treatment of bifurcation damage, the operator should initially pass or catheterize the vessel with the bifurcation damage with a guide catheter and connect the guide catheter to the appropriate Y connector 40 for bifurcation. Subsequently, we should fix the Y connector 40 in bifurcation to the base or platform 11, firstly fixing its more distal portion and then its proximal portion or, alternatively, fixing is proximal portion and then its distal portion (as shown by FIGS. 4 to 6), so that said connector 40 is stabilized and with its two inlets 42 and 43 related and aligned, respectively, to fixers 17, 17′ and 18,18′ of the metal guides and balloon catheters.

After fixing the connector 40, the operator should pass a metal guide to the main vessel by means of the inlet 42 of the Y connector 40, which is more aligned with the guide catheter. At that point, and before starting to pass the guide through the obstruction, the operator fixes the directing device (not shown) of the metal guide, as commonly used in interventions, at the zero point of the millimeter ruler 20 (please refer to FIG. 10A) as printed at the line of the fixers 17, 17′, thus marking the start of the damage. Continuing with the advance of the metal guide and after reaching the end of the obstruction, the operator makes the guide directing device abut the ruler 20 and evaluates the linear displacement of the metal guide, from which it is possible to make a precise estimative of the extension of the damage. Based on that value, the operator can then choose the size of the balloon or stent to be used more precisely and safely.

The advancement of the metal guide follows to reach the distal bed of the vessel and then the operator fixes the guide to the pair of fixers 17 and 17′ which is aligned; the operator then passes a second metal guide to the side or secondary branch through the other inlet 43 of the Y connector, which is more crosswise with the guide catheter and, when reaching the distal bed of said vessel, the operator is fixed in its pair of fixers 18, 18′, similarly to the above description. The operator now has the metal guides as identified, positioned and fixed, as well as a faithful estimative of the extension of the estimated damage.

Dilatations are then made with balloon catheters at the obstructions of the main and side vessels. To perform this skill, the operator needs to put and position two balloons at the level of obstructions and insufflate them separately or simultaneously, to then release one or two endovascular prostheses or stents. With that purpose, the operator releases the guide of the main vessel of fixers 17 and 17′ and passes the balloon catheter over the metal guide and, soon after passing the more proximal portion of the balloon catheter, the operator fixes again the metal guide to said fixers 17, 17′ and then pushes the balloon catheter over the metal guide and, after positioning the balloon at the obstruction level, the operator also fixes the balloon catheter to the fixers 17, 17′ of the corresponding guide, so that they do not go back or forth when a second balloon catheter is introduced into the side or secondary vessel.

When a second balloon is required, the operator performs the same procedures as for the first one, releases the metal guide of the fixers 18, 18′, passes the balloon catheter, again fixes the guide into the corresponding fixers 18, 18′ and pushes the balloon catheter until the level of obstruction. In that moment, the operator can handle, at the same time, both balloon catheters and locate them more safely and quickly.

After balloon insufflations and de-insufflations, balloon catheters are pulled, keeping fixed metal guides. To take off the balloon catheter, the operator slowly pulls the balloon catheter until its proximal portion reaches the inlet 42 of the Y connector.

After the injection of contrast agent by the route 41 of the Y connector 40, the operator will decide which skill will be used to treat bifurcation obstructions.

In case the operator decides to implant endovascular prosthesis or stents, he/she should follow the same steps as previously disclosed for balloon catheters, since those prosthesis are fixed or clipped to the balloon of balloon catheters. Therefore, the operator releases the guide of the main vessel from fixers 17, 17′ and passes the balloon catheter with the endovascular prosthesis over the metal guide, fixes it again to the fixers 17, 17′ and pulls the balloon catheter over the guide until the level of obstructions and, after the balloon is positioned with the endovascular prosthesis, the operator insufflates the balloon, releasing the stent at the place of obstruction.

When the second balloon catheter is required at the side or secondary vessel, the operator makes the same procedure as the first one, i. e. he/she releases the guide from the fixers 18, 18′, passes the balloon catheter, fixes it again in the same fixers, pushes the balloon until the level of obstruction and insufflates it alone or simultaneously with the balloon from the main vessel.

In case the operator still desires to insufflate the balloons or release the stents at the same time, he/she may simultaneously handle both balloon catheters with stents and position them more quickly and safely.

To take off the balloon catheter, the operator slowly pulls the balloon catheter until its proximal portion reaches the inlet 42 or 43 of the Y connector.

As we can conclude from the above description, the use of any one of the devices 1, 2, 3 or 4, as disclosed above, makes the surgical procedure become easier, faster and safer for the patient, also reducing the quantity of X rays to which patients and doctors are exposed.

Concerning the operator, devices 1, 2, 3 or 4 facilitate handling of guides and their respective catheters, facilitating the identification of each one, thus avoiding eventual prejudicial crossing between guides and catheters, and especially reducing the risk of displacements during the procedure.

1. A device to fix metal guides and balloon catheters to make medical procedures in percutaneous vascular interventions comprising a flat base having a proximal end and a distal end, wherein respective fixing means on said narrow proximal end project to support and keep a Y connector in position and at least one aligned pair of fixers on said distal end projects, and each pair of fixers is also aligned with the respective inlets to the main vessel and to secondary or side vessels.

2. The device of claim 1, wherein the fixing means of the Y connector comprise a jaw, in the form of a full prismatic element and provided in its upper part with two opposed holding arms, said arms limit a seat with similar shape to the external cylindrical form of the proximal portion of the Y connector and pins, in the form of converging prismatic structures, having a recess able to couple the distal portion of the Y connector.

3. The device of claim 1, wherein a respective orifice is located below each one of the inlets of the Y connector for the flow of blood.

4. The device of claim 1 wherein the fixer is a trunk-pyramidal structure made of a rigid material, projecting from the base and having on its upper portion a graft of liable material, and provided with a lengthwise gutter defining a seat to receive and retain a metal guide, a balloon catheter or a balloon catheter with stent.

5. The device of claim 4, wherein the graft has a prismatic shape with a crosswise half-circled section.

6. The device of claim 1, further comprising at least a millimeter ruler located on the base and at the alignment axis between the pairs of fixers.

7. The device of clam 6, wherein the millimeter ruler is printed on the base.

8. The device, particularly for the treatment of bifurcation damage, of claim 1, wherein the proximal end of the base supports, by fixing means, a Y connector comprising an inlet for the main vessel and an inlet for a secondary or side vessel, and the pair of fixers is aligned with said inlet to the main vessel and the pair of fixers is aligned with said inlet to a secondary or side vessel.

9. The device of claim 8, further comprising a V-shaped discontinuity formed between the support regions of the fixers and, each one defining a projection or leg from the central region of the base of the device.

10. The device, particularly for the treatment of simple damage or angioplasty, of claim 1, characterized by the fact that the proximal end of the base supports, by fixing means, a Y connector comprising an inlet to the main vessel, wherein the fixers are aligned with said inlet to the main vessel.

11. The device, especially for the treatment of trifurcation damage, of claim 1, wherein the proximal end of the base supports by the fixing means a Y connector comprising an inlet to the main vessel and two inlets to at least one of the secondary or side vessels, and the pair of fixers is aligned with said inlet to the main vessel, the pair of fixers is aligned with said inlet to a secondary or side vessel and the pair of fixers is aligned with said inlet to a secondary or side vessel.
